(19) United States
(12) Patent Application Publication
(10) Pub. No.: US 2004/0220619 A1
(43) Pub. Date: Nov. 4, 2004

(54) Method of inhibiting the proliferation of vascular smooth muscle cells

(76) Inventors: Chuwa Tei, Kagoshima (JP); Koji Orihara, Kagoshima (JP)

Correspondence Address: SHERIDAN ROSS PC, 1560 BROADWAY, SUITE 1200, DENVER, CO, 80202

(21) Appl. No.: 10/482,516

(22) PCT Filed: Jun. 26, 2002
(86) PCT No.: PCT/JP02/06403

(30) Foreign Application Priority Data
Jun. 27, 2001 (JP) 2001-235607

(51) Int. Cl. A61N 1/00

(52) U.S. Cl. 607/1

(57) ABSTRACT

The hyperthermic treatment is applied to synthesis vascular smooth muscle cells that have undergone the growth stimulation, whereby only the abnormal growth is selectively inhibited without having an adverse effect on the surrounding normal cells. The timing of the hyperthermic treatment is preferably after the growth stimulation, more preferably 2 hours after the growth stimulation. The hyperthermic treatment temperature is preferably in the range of from 42° C. to 44° C., and 43° C. is optimal. The hyperthermic treatment time is preferably in the range of from 90 to 180 minutes, and 120 minutes is optimal. Outside the ranges of the hyperthermic treatment conditions, the growth inhibition effect might be insufficient, the normal contraction type smooth muscle cells might be brought to cell death, or the growth of the vascular endothelial cells might be inhibited. The hyperthermic treatment is a safe therapy with less pain to patients, which can be clinically applied as a method for hindering restenosis after coronary lesion angioplasty.

### TECHNICAL FIELD

The present invention relates to a method for inhibiting growth of vascular smooth muscle cells. More specifically, it relates to a method for inhibiting growth of vascular smooth muscle cells by a cell culture engineering process.

### BACKGROUND ART

In recent years, percutaneous transluminal coronary angioplasty (PTCA) has been widespread as a method for treating ischemic heart diseases. In PTCA, a balloon catheter is percutaneously inserted into blood vessels without abdominal section to dilate a coronary stenosis site for treatment. This has been evaluated as an excellent therapy with less pain to patients.

Nevertheless, according to Adelman A G et al. “A comparison of directional atherectomy with balloon angioplasty for lesions of the left anterior descending coronary artery, N Eng J Med. 329 228-233(1993) Comment”, restenosis occurs in the treated site at a high rate of approximately 30% or more of actual cases. When, for example, stent indwelling is employed in this restenosis, it is effective against acute reclogging to some extent. However, restenosis which undergoes chronic progression by participation of local inflammation and subsequent new tunica intima hyperplasia and vascular remodeling has been still unresolved.

The coronary artery comprises a three-layer structure of tunica intima, tunica media and tunica externa. The tunica intima comprises endothelial cells, and the tunica media comprises smooth muscle cells. In the progression of restenosis that takes place after PTCA or stent indwelling, hyperplasia of smooth muscle cells in a subendothelial cavity is deemed to be one of the major causes. According to studies by molecular biological methods in recent years, it is deemed that intervention therapy of coronary lesions causes transformation of normal contraction type smooth muscle cells constituting the tunica media into synthesis type cells, whereby these cells acquire a migration potency to repeat invasion into a subendothelial cavity and growth division, which triggers new tunica intima hyperplasia.

This method for preventing restenosis is roughly classified into a method using various drugs and a cell culture engineering method. Although the former has been long used, clinically effective drugs have not been found as yet. Recently, the method for inhibiting growth of smooth muscle cells by administration of interferons has been tried (JP-A-9-151137), however, it causes strong side effects which give great pain to patients, whereas the therapeutic effect is not clear.

The cell culture engineering process can include a genetic engineering method and a radioactive method. As the genetic engineering method, for example, “Santa M et al Fas ligand gene transfer to the vessel wall inhibits neointim a formation and overrides the adenovirus-medicated T cell response Proc Natl Acad Sci USA. 95 1213-1217(1998)”, has been known as the method for inhibiting the new tunica intima hyperplasia, however, which is far from clinical application.

Meanwhile, the radioactive therapy has also attracted interest in the field of this restenosis prevention. With respect to the radiation method, a method using a catheter (teirstein P S et al A double blinded randomized trial of catheter-based radio-therapy to inhibit restenosis following coronary stenting N Engl J Med. 336 1697-1703 (1997)), and a method in which a radioactive stent is indwelled are employed (Waksman, R. et al., Clinical and angiographical follow-up after implantation of a 6-12?Ci radioactive stent in patients with coronary artery disease Eur Heart J. 22 669-675 (2001)) have been known. These methods have been clinically applied, but have involved new problems such as deficient regeneration of vascular endothelial cells and restenosis on opposite ends of the stent at high frequency.

A report using hyperthermic treatment at restenosis after the coronary arteries formation, David G. Neschis et al., Thermal preconditioning before rat arterial balloon injury; limitation of injury and sustained reduction of intimal thickening Arterioscler Thromb Vasc Biol. 18 120-126 (1998), has been done. The fact that decrease of restenosis was recognized when hyperthermic treatment conducted at 43° C. for 15 minutes to the whole body of a rat at 6 hours before the coronary arteries formation has been written in this report, however, the appropriate condition for inhibiting the overgrowth and for the hyperthermic treatment are unclear in this report.

It is an object of the invention to provide a method for inhibiting growth of smooth muscle cells, which comprises selectively inhibiting abnormal growth of smooth muscle cells shifted to the synthesis type by less damaging cells constituting the tunica intima and tunica media.

Another object of the invention is to provide a method for inhibiting growth of vascular smooth muscle cells which method is high in medical utility of preventing restenosis that occurs in intervention therapy of coronary lesions, which is clinically performed safely and easily with less pain to patients.

### DISCLOSURE OF THE INVENTION

A method for inhibiting growth of vascular smooth muscle cells in the invention, by which the foregoing objects have been achieved, is characterized by comprising subjecting the vascular smooth muscle cells which has responded to growth to the hyperthermic treatment.

The timing of applying the hyperthermic treatment can be arbitrarily selected from 12 hours before growth stimulation of the smooth muscle to 15 hours after the stimulation including the point of time of the stimulation. It is preferably after the growth stimulation, more preferably 2 hours after the growth stimulation.

The hyperthermic treatment temperature is not absolutely specified relative to the treatment time. The temperature is preferably from 42° C. to 44° C., and 43° C. is optimal. When it is less than 42° C., the effect of growth inhibition is insufficient. When it exceeds 44° C., strong inhibition of grown smooth muscle cells is observed, but normal contraction type smooth muscle cells having no growth potency are also brought to cell death, and growth inhibition of vascular endothelial cells also starts.

The hyperthermic treatment time is preferably in the range of from 90 to 180 minutes, and 120 minutes is optimal. When the treatment time is less than 90 minutes, no desired effect of growth inhibition is identified. Even though it exceeds 180 minutes, only approximately the same effect of growth inhibition as given for 120 minutes is identified. In addition, an adverse effect might be exerted on growth of normal contraction type vascular smooth muscle cells and vascular endothelial cells having no growth potency.

In view of the foregoing, it is most preferable that the hyperthermic treatment at 43° C. for 2 hours is applied to vascular smooth muscle cells after the lapse of 2 hours from the growth stimulation thereof.

### BRIEF DESCRIPTION OF THE DRAWINGS

FIG. 1 is a graph showing a growth inhibition effect that the hyperthermic treatment gives vascular smooth muscle cells at the logarithmic growth phase.

FIG. 2 is a graph showing a growth inhibition effect that the hyperthermic treatment just after the growth stimulation gives quiescent smooth muscle cells of a cell culture experimental system.

FIG. 3 is phase-contrast micrographs showing a growth inhibition effect that the hyperthermic treatment 2 hours after the growth stimulation gives quiescent smooth muscle cells of a cell culture experimental system.

FIG. 4 is a graph showing a growth inhibition effect that the growth stimulation after the hyperthermic pretreatment gives quiescent smooth muscle cells of a cell culture experimental system.

FIG. 5 is graphs showing a growth inhibition effect that the hyperthermic treatment temperature and the timing of the hyperthermic treatment after the growth stimulation give quiescent smooth muscle cells of a cell culture experimental system.

FIG. 6 is graphs showing a growth inhibition effect that the hyperthermic treatment time gives quiescent smooth muscle cells of a cell culture experimental system in the hyperthermic treatment after the growth stimulation.

FIG. 7 is a graph showing a growth inhibition effect that the hyperthermic treatment gives quiescent smooth muscle cells of a cell culture experimental system in place of normal contraction type smooth muscle cells.

FIG. 8 is graphs showing a growth inhibition effect that the hyperthermic treatment gives bovine aortic endothelial cells of a cell culture experimental system in place of vascular endothelial cells.

FIG. 9 is graphs showing, in terms of a change of cell cycle with time, results of analyzing a cellular DNA content of smooth muscle cells in the hyperthermic treatment after the growth stimulation using a flow cytometer.

FIG. 10 is graphs showing, in terms of a change of cell cycle with time, results of analyzing a cellular DNA content of smooth muscle cells in the hyperthermic treatment without the growth stimulation using a flow cytometer.

FIG. 11, FIG. 12, FIG. 13 and FIG. 14 are micrographs showing results of Giemsa staining of smooth muscle cells after the hyperthermic treatment.

FIG. 15 is photographs showing results of DNA ladder detection of smooth muscle cells after the hyperthermic treatment.

FIG. 16, FIG. 17 and FIG. 18 are optical micrographs showing results of TUNEL staining of quiescent smooth muscle cells after the hyperthermic treatment.

FIG. 19, FIG. 20, FIG. 21, FIG. 22 and FIG. 23 are graphs showing annexin V-FITC test results of smooth muscle cells after the hyperthermic treatment.

### BEST MODE FOR CARRYING OUT THE INVENTION

The best mode for carrying out the invention is described in detail below by referring to the drawings.

One of the major causes of restenosis which takes place after actual coronary angioplasty and characterized by chronic progression is abnormal hyperplasia of vascular smooth muscle cells in the vascular endothelial cavity. Release of various cell growth factors and cytokines from platelets agglomerated by damage of vascular endothelial cells owing to a mechanical pressure accompanied by ballooning or the like or from necronized cells causes transformation of contraction type cells to synthesis type cells of the vascular smooth muscle in an early stage after the treatment, which leads to hyperplasia of smooth muscle cells.

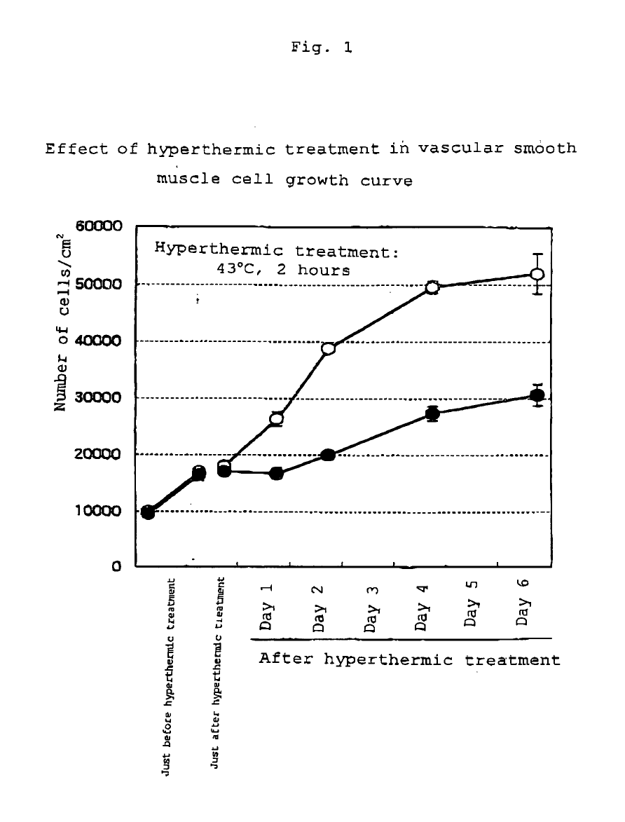

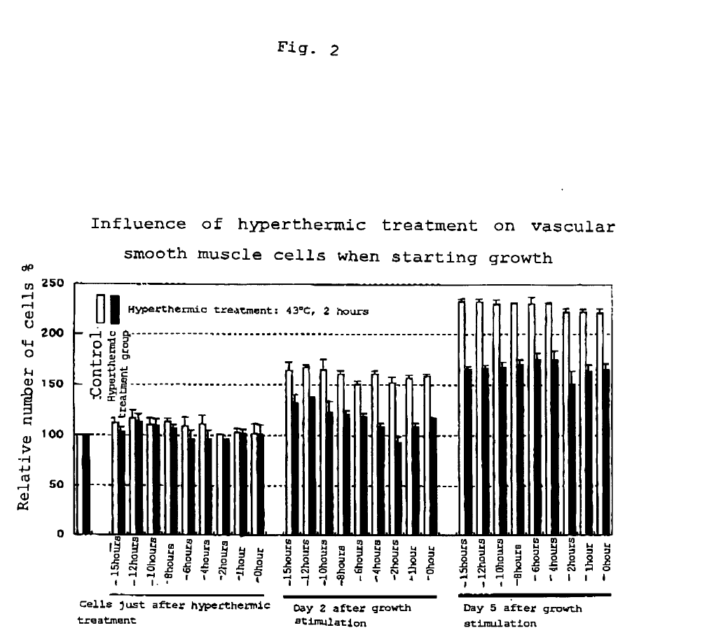

When, for example, the hyperthermic treatment at 43° C. for 2 hours is applied to synthesis type vascular smooth muscle cells (VSMCs) at the logarithmic growth phase, the cells reach a nearly quiescent state from just after the treatment to day 1 as shown in FIG. 1, and the growth is remarkably inhibited from day 2 onward. This is quite contrasted with the fact that control cells at the logarithmic growth phase without the hyperthermic treatment continue the growth.

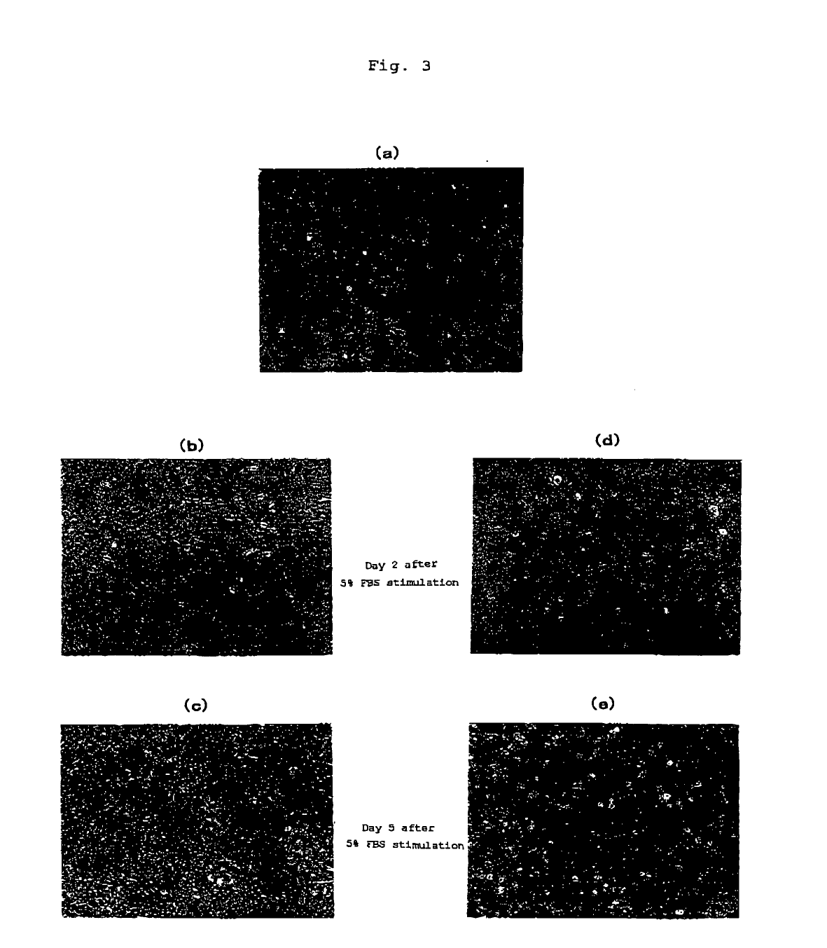

Regarding quiescent smooth muscle cells of a cell growth experimental system in place of normal contraction type smooth muscle cells constituting the vascular tunica intima, the timing of the hyperthermic treatment in the invention can be arbitrarily selected from 12 hours before the growth stimulation to 15 hours after the growth stimulation as shown in FIGS. 2 and 3. From the standpoint of the growth inhibition effect, as is clear from FIG. 2, the timing is preferably up to 15 hours after the growth stimulation including the point of time of the growth stimulation of the smooth muscle. Even though the hyperthermic treatment is performed at any point of time after the growth stimulation, the cells subjected to the hyperthermic treatment shows the significant cell growth inhibition effect in comparison to the control cells without the hyperthermic treatment. With respect to the growth inhibition effect, it is not a phenomenon that caused by the decrease number of cells(cell death) but caused by inhibiting the increase number of cells for 2 days after growth stimulation, and the effect is still remarkable for 5 days long term after the growth stimulation.

According to the observation on day 2 after the growth stimulation, the hyperthermic treatment at 2 hours after the growth stimulation shows the highest growth inhibition effect. In the hyperthermic treatment at the other points of time, the number of cells is still increased slightly. The optimum timing of the hyperthermic treatment after the growth stimulation in a model of a cell culture experimental system is 2 hours after the growth stimulation.

This growth inhibition effect is also found from the micrographs in FIG. 3. For example, according to the observation on day 5 after the growth stimulation, the control cells without the hyperthermia treatment reach a high cell density state indicating a decrease in growth potency owing to cell contact hindrance as shown in FIG. 3(e). Meanwhile, the cells subjected to the hyperthermia treatment are still low in cell density at this point of time, and delay of growth is clearly observed therein.

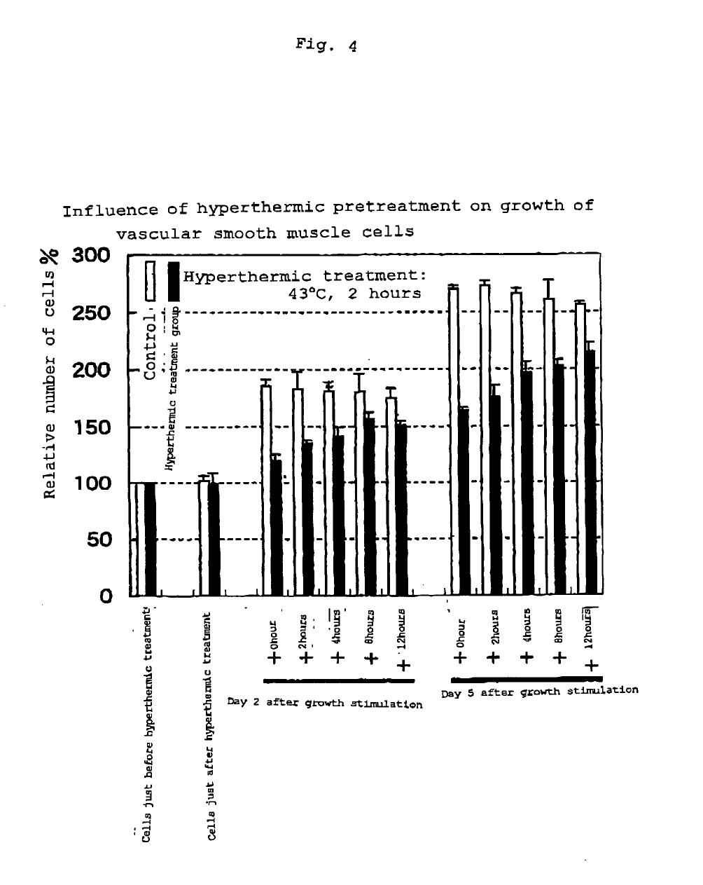

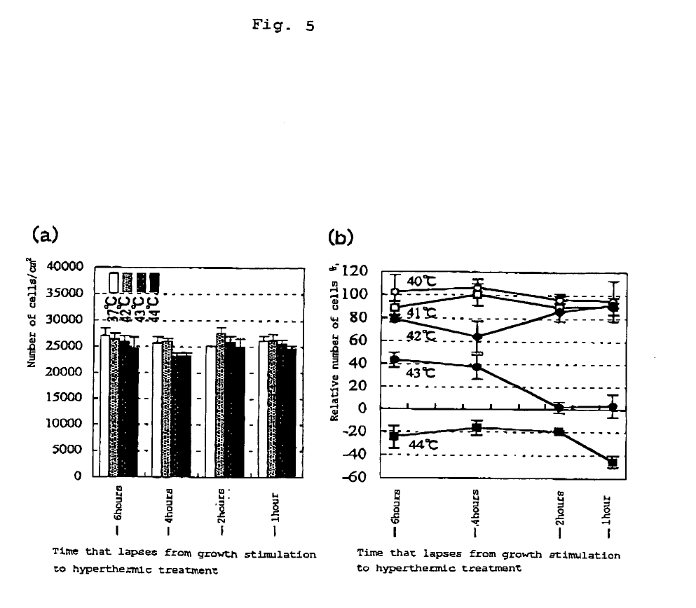

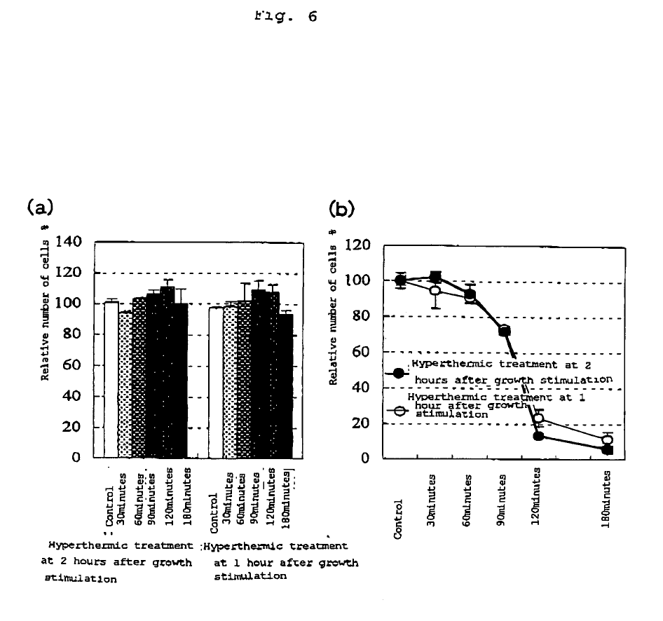

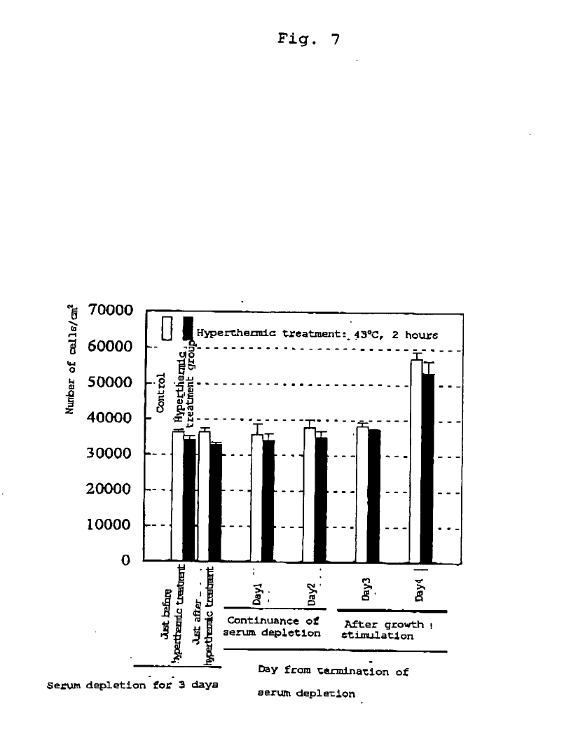

On the contrary, when quiescent smooth muscle cells are subjected to the hyperthermic pretreatment (thermal preconditioning) and then to the growth stimulation, the cells to which the hyperthermic treatment and the growth stimulation have been applied at the same time show the highest growth inhibition effect as shown in FIG. 4. The earlier the growth stimulation after the hyperthermic treatment, the better.

Regarding the relation between the hyperthermic treatment temperature and the timing of the hyperthermic treatment, as shown in FIG. 5(a), the cell growth inhibition effect is observed so long as the hyperthermic treatment temperature is at least 42° C. As is clear from FIG. 5(b), the decrease in number of cells is observed at 42° C. in the hyperthermic treatment after 4 hours from the growth stimulation, but the rate thereof is only approximately 35%. Meanwhile, the remarkable growth inhibition effect is observed at 43° C. regardless of the timing of the hyperthermic treatment. Especially in the hyperthermic treatment at 1 hour and 2 hours after the growth stimulation, the maximum growth inhibition effect is observed. At 44° C., the number of cells is smaller than the number of cells of the control sample before the growth stimulation in the hyperthermic treatment at any timing. Conversely, when the temperature reaches 44° C., it is suggested that the growth inhibition effect is attributable to the loss of cells by cell death.

In view of these points, the growth treatment temperature is most preferably 43° C. The timing of the hyperthermic treatment after the growth stimulation is most preferably 2 hours after the growth stimulation.

Meanwhile, regarding the preferable hyperthermic treatment time in the invention, as is clear from FIG. 6, the significant growth inhibition effect is confirmed from the treatment time of 90 minutes in the hyperthermic treatment at both 1 hour and 2 hours after the growth stimulation at the hyperthermic treatment of 43° C. The effect close to the growth stop is confirmed from at least 120 minutes. Accordingly, the optimum hyperthermic treatment time is 2 hours.

Assuming that the hyperthermic treatment of the invention is clinically applied to prevention of restenosis, heat is transmitted throughout the three-layer structure of the vascular wall even though the hyperthermic treatment is applied to desired parts of blood vessels. When an adverse effect of heat is exerted on normal contraction type smooth muscle cells constituting the tunica media of the vascular wall and having no growth potency, the hyperthermic treatment of the invention is hardly applied to the clinical therapy. To put it in another way, when the hyperthermic treatment is used to prevent the growth of the smooth muscle cells that grow abnormally by cytotoxic treatment, the growth inhibition effect in the hyperthermic treatment has to have a cell growth state selectivity depending on the presence or absence of a growth potency in the application range.

The cell growth state selectivity of the growth prevention effect in the hyperthermic treatment is clear from FIG. 7. That is, in the quiescent smooth muscle cells of the cell culture experimental system in place of the contraction type smooth muscle cells constituting the tunica media of the vascular wall, approximately the same cell growth as in the control sample without the hyperthermic treatment can be identified when observed just after the hyperthermic treatment at 43° C. for 2 hours, on day 1 and day 2 after the serum depletion culture subsequent to the hyperthermic treatment, and on day 3 and day 4 with the growth stimulation applied.

Upon comparing this with the growth inhibition effect in the smooth muscle cells at the logarithmic growth phase in FIG. 1, it is found that the hyperthermic treatment selectively shows the growth inhibition effect to the smooth muscle cells at the logarithmic growth phase but does not influence at all the cell growth of the quiescent smooth muscle cells.

Meanwhile, in the vascular endothelial cells, the damage of the endothelial cells inevitably occurs after the colonary angioplasty. Regeneration of the endothelial cells after the damage is an important repair reaction that acts to inhibit the growth of the smooth muscle in the subendothelial cavity. However, when the growth of the endothelial cells is also inhibited along with the growth of the smooth muscle cells by the hyperthermic treatment, the hyperthermic treatment for inhibiting restenosis is lacking in certainty and stability.

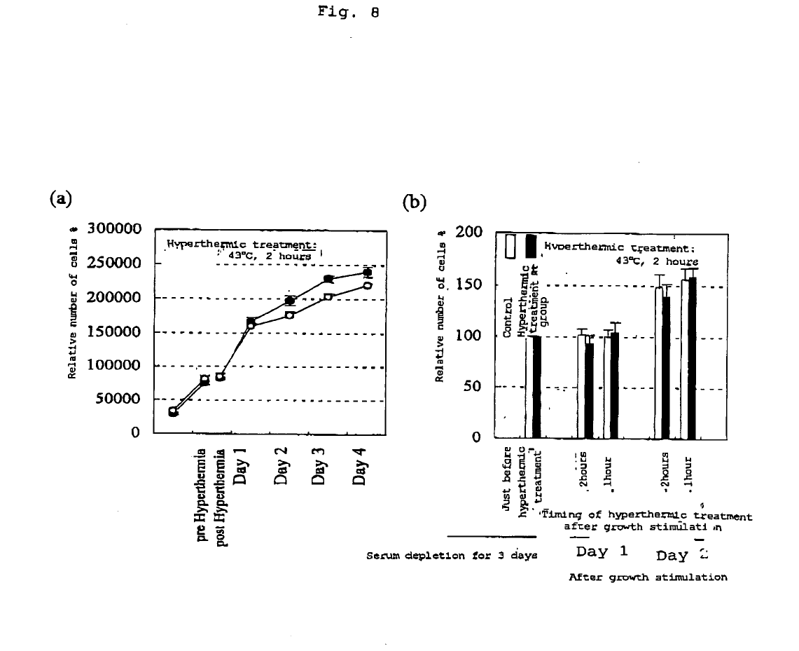

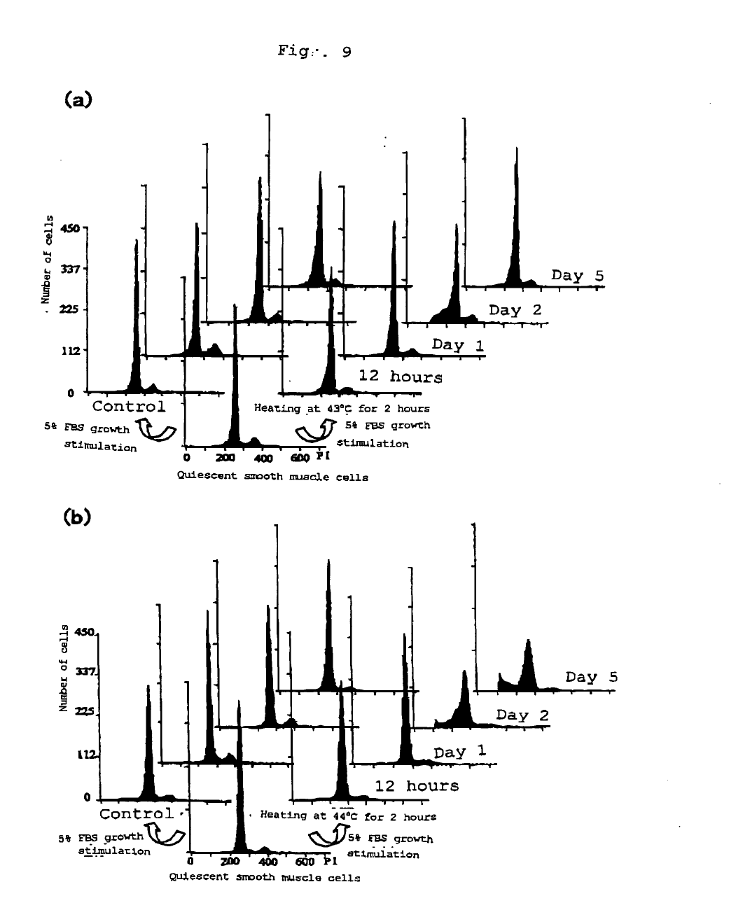

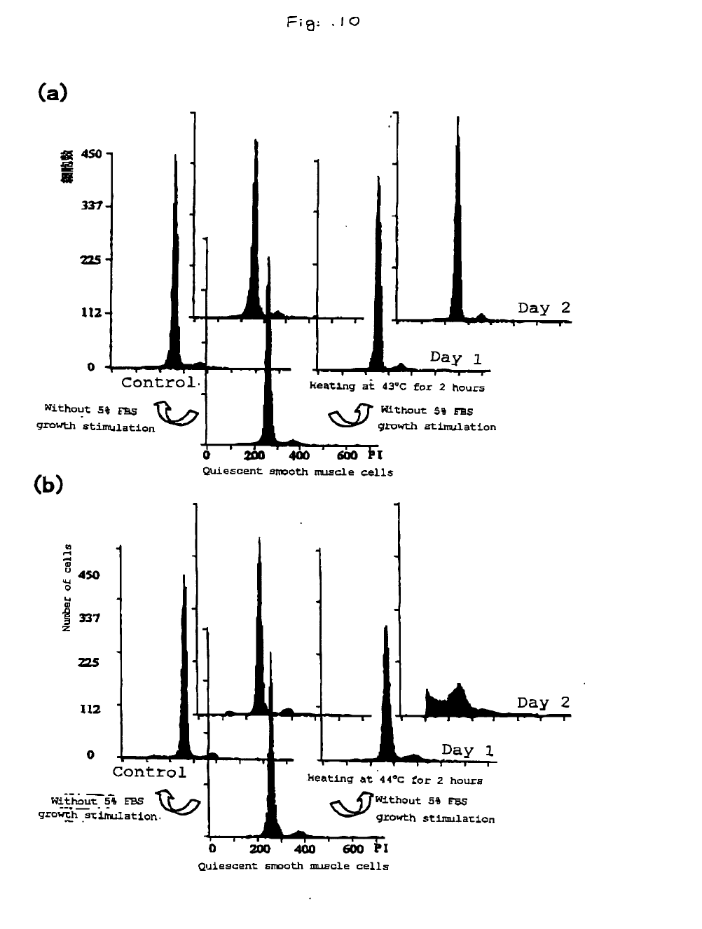

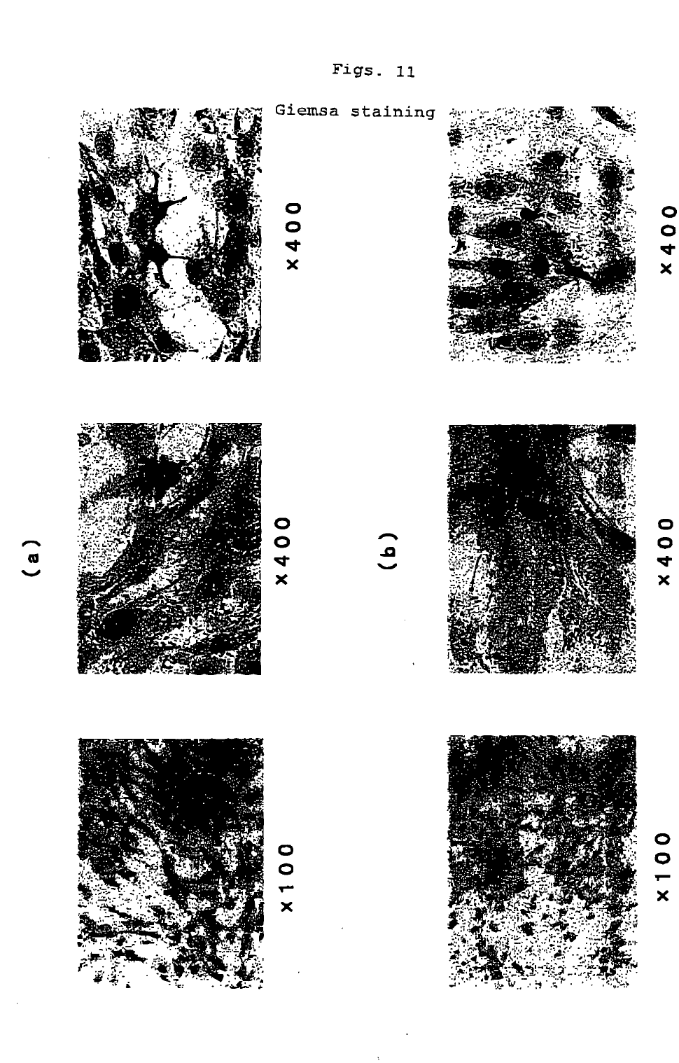

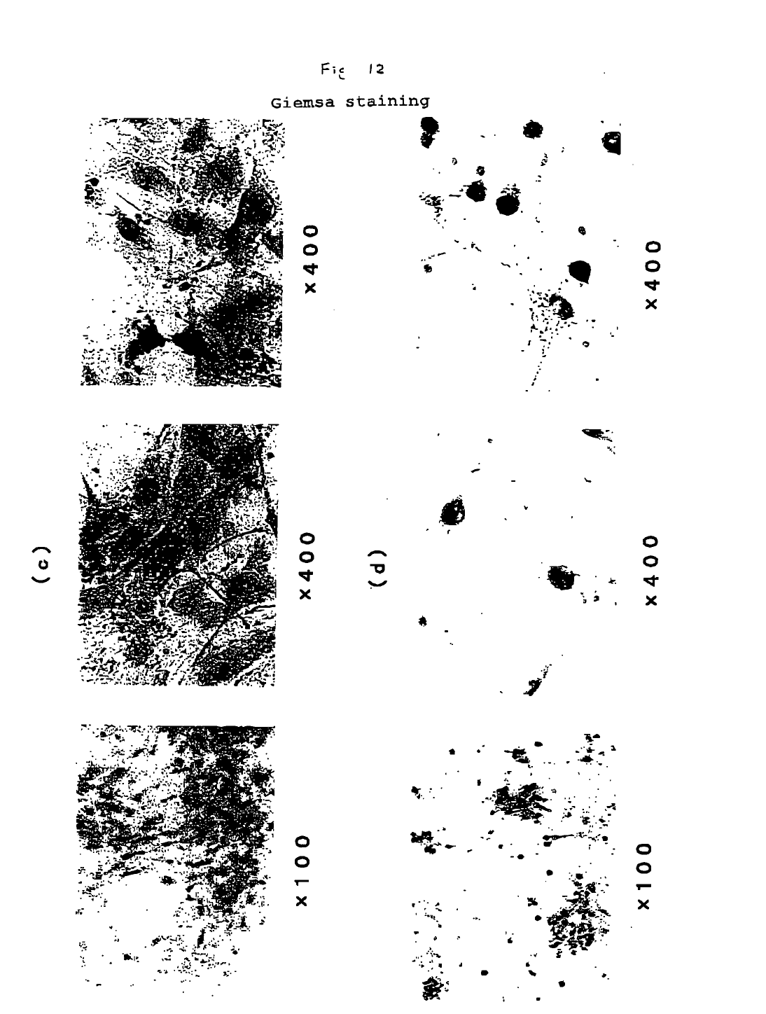

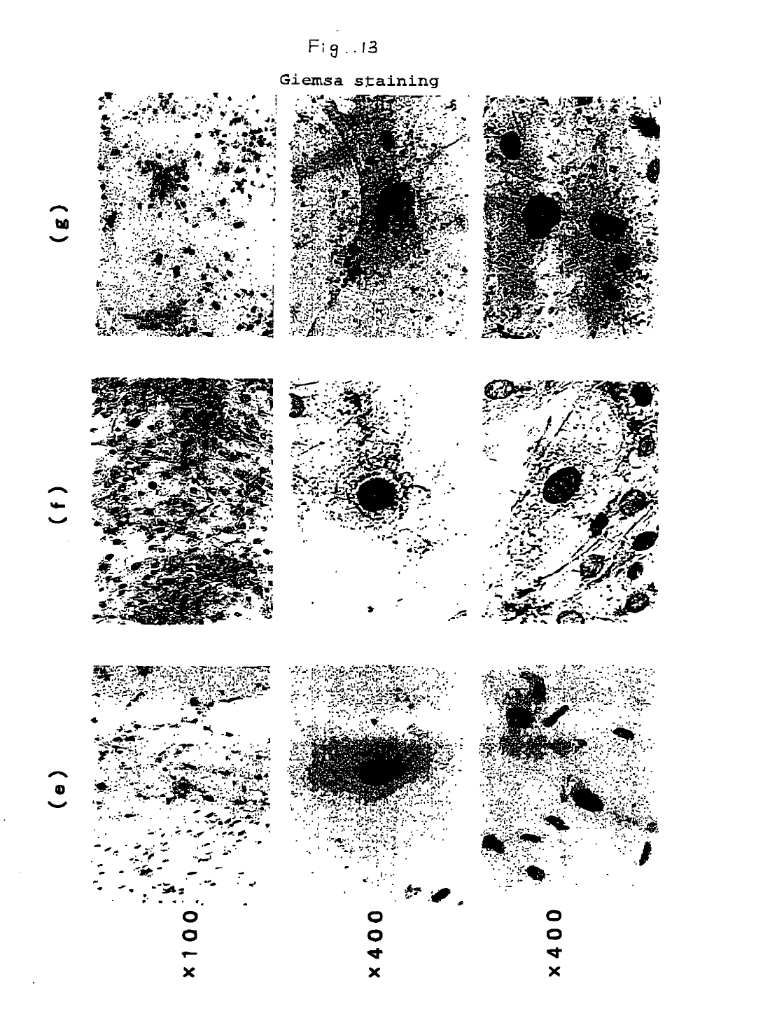

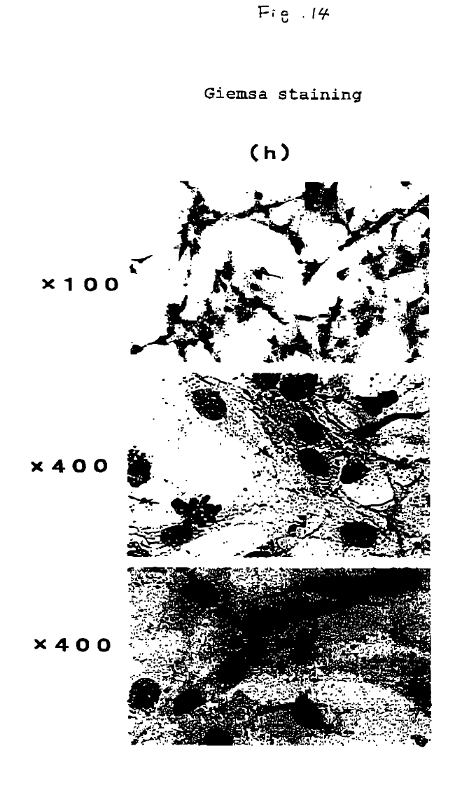

As a model of the cell culture experimental system, bovine aortic endothelial cells (BAECs) close to the vascular endothelial cells were subcultured, and BAECs at the logarithmic growth phase in which the growth rate was highest on days 4 and 5 after the cell inoculation were subjected to the hyperthermic treatment at 43° C. for 2 hours. Consequently, as shown in FIG. 8(a), the growth of cells is approximately the same as that of control cells without the hyperthermic treatment at any point of time of days 1 to 4 after the hyperthermic treatment, and no adverse effect of the growth inhibition by the hyperthermic treatment is confirmed. This is quite contrasted with FIG. 1 showing the growth inhibition effect by the hyperthermic treatment of synthesis type smooth muscle cells at the logarithmic growth phase.

Likewise, quiescent bovine aortic endothelial cells (quiescent VSMCs) obtained by subjecting BAECs at the logarithmic growth phase to serum depletion culture for 3 days was subjected to the hyperthermic treatment at 43° C. for 2 hours, at 1 hour and 2 hours after the growth stimulation. As a result, the cell growth rate is approximately the same as that of the control cells without the hyperthermic treatment in the observation on both day 1 and day 2 after the growth stimulation as shown in FIG. 8(b), and no adverse effect of the growth inhibition by the hyperthermic treatment is found.

These facts reveal that the hyperthermic treatment of the invention has the cell selectivity and the cell growth state selectivity and selectively shows the growth inhibition effect only to the synthesis type smooth muscle cells at the logarithmic growth phase, but does not influence at all the quiescent smooth muscle cells in place of the normal contraction type smooth muscle cells of the cell culture experimental system or BAECs at the logarithmic growth phase and quiescent BAECs in place of the vascular endothelial cells. Accordingly, when the hyperthermic treatment of the invention is clinically applied, the cell growth of the synthesis type smooth muscle transformed is selectively inhibited but there is no possibility of inhibiting the growth regeneration of the normal contraction type smooth muscle cells of the vascular tunica media or the vascular endothelial cells. The stenosis after the coronary angioplasty can be inhibited by the hyperthermic treatment of the invention safely and effectively.

The reason why the hyperthermic treatment inhibits the growth of the synthesis type smooth muscle cells at the growth phase is not necessarily clear. However, the influence of heat shock proteins (HSPs) can be considered. The effect of inhibiting agglomeration of soluble proteins by heat shock has been so far known, and it has been considered as one mechanism of a heat resistance of cells. It has been nowadays found that HSPs participate in a wide portion of a normal physiological function called molecular chaperone. Even in the absence of the heat shock, HSPs act as an important multifunctional regulatory factor through a protein structure changing potency. From this standpoint, it is presumed that the hyperthermic treatment of cells allows further induction of HSPs or migration thereof into the nucleus which leads to the cell growth inhibition of the vascular smooth muscle cells.

At any rate, assuming that the hyperthermia is applied as a method for preventing restenosis after vascular angioplasty, the phenomenon of cytological change of the cell growth inhibition by the hyperthermic treatment of the invention is a problem that cannot be overlooked in view of the safety of the hyperthermia.

As a result of analyzing an intracellular DNA content with a flow cytometer, this phenomenon is mostly ascribable to the growth stop at G1 phase of the cell cycle, and partially (approximately 15%) ascribable to appearance of a cell cycle sub-G1 population which means destruction (cell death) of a cellular DNA.

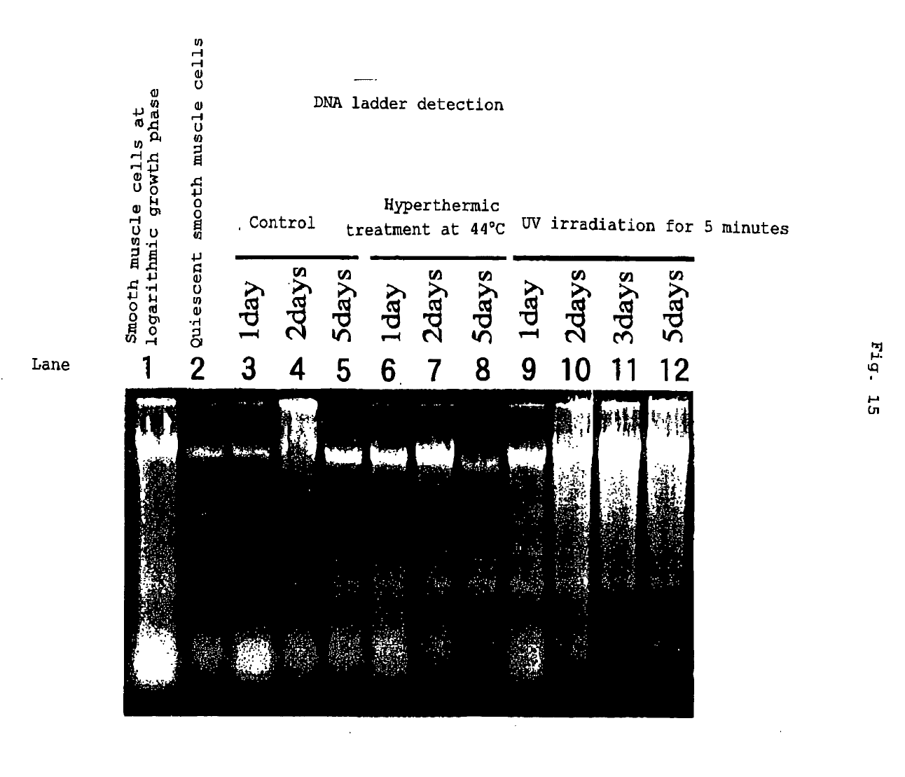

That is, as shown in FIG. 9(a), when the quiescent smooth muscle cells are subjected to the hyperthermic treatment at 43° C. for 2 hours, the number of cells shifted to S phase and G2/M phase on day 1 after the growth stimulation is overwhelmingly small in comparison to the control cells without the hyperthermic treatment, showing a typical pattern of GI phase stop phenomenon. In contrast, even when the hyperthermic treatment is applied to the cultured vascular endothelial cells under the same conditions, there is no function of inhibiting the cell growth. No cell death is observed either in the quiescent smooth muscle cells with the growth stopped.

This suggests that when the cell growth is inhibited on the smooth muscle cells that have undergone the growth stimulation by the hyperthermic treatment of the invention, the cell selectivity is provided on the cell cycle G1 stop phenomenon that takes place on most of the cells and the cell selectivity and the cell state selectivity are provided on the cell death induced in part of the cells.

Whether the cell death shown in the cell cycle sub-G1 population is ascribable to necrosis or apoptosis has quite an important significance when assuming the clinical application of the hyperthermia. That is, when it is ascribable to necrosis, an inflammation reaction takes place which might lead to further growth stimulation of the smooth muscle cells constituting the vascular tunica media by invasion of reactive lymphocytes. Meanwhile, when it is ascribable to apoptosis, no such secondary inflammation takes place, and a safe and effective therapy is provided.

According to the micrographs given by a Giemsa staining method as shown in FIG. 11, reduction in size of a whole cell, agglomeration and fragmentation of a nucleus which are typically observed in apoptosis are identified in approximately 15% of the cells. In the lane photographs given by a DNA ladder detection method in FIG. 12, the DNA extract from the cells subjected to the hyperthermic treatment shows fragmentation (DNA ladder) in a nucleosome unit. The micrographs given by the TUNEL staining method as shown in FIG. 13 indicate TUNEL-positive. From these results, this cell death is presumably based on the apoptosis mechanism.

However, according to the analysis with a flow cytometer using annexin V-FITC/PI, more than half of the apoptosis cell group induced by subjecting the quiescent smooth muscle cells to the hyperthermic treatment at 43° C. or 44° C. for 2 hours after 2 hours from the growth stimulation is negative in the detection of the cell membrane surface exposure of the annexin V binding site (phosphatidylserine) as shown in FIG. 14. This is quite a peculiar result. When this is generally judged from the foregoing tests, the mode of the cell death can be presumed to be safe apoptosis.

In view of the foregoing, the hyperthermic treatment of the invention is safe and effective in the clinical application as a therapy of preventing the restenosis after the coronary angioplasty when the heating conditions are strictly determined and controlled.

As a heating method in the clinical application, a dielectric heating method, an induction heating method, an insertion heating method, an implantation heating method and the like can be employed. In the dielectric heating method, two electrodes are put on the surface of the body, and heating is performed by passing a current between both electrodes. In the induction heating method, a current is passed through a cylindrical coil to generate a magnetic field, and heating is performed with the induction current given by the magnetic field. In the insertion heating method, an electrode is inserted into the body, and heating is performed by passing a current while the electrode is put in an affected part. In the implantation heating method, a heating unit is implanted in the vicinity of an affected part, and heating is performed by feeding a heat energy from outside the body.

As a heating medium, infrared rays, microwaves, high-frequency waves, ultrasonic waves and the like can be used. Microwaves excellent in deep transmission, non-invasion and local heat generation in tissues are especially preferable.

### EXAMPLE 1

Vascular smooth muscle cells (VSMCs) were collected from the tunica media of the rat breast aorta, and subjected to primary culture by an explanation method. VSMCs subjected to the primary culture were subcultured in an incubator using a culture flask. The incubator was set at a temperature of 37° C., and filled with humid air containing 5% carbon dioxide. In the subculture, a cell liquid culture was replaced every three days. When the culture cell density became high, the cells were recovered with EDTA-trypsin, and inoculated in another flask. The cell growth liquid medium contained 10% fetal bovine serum (FBS), 100 units/ml of penicillin and 100 mg/ml of streptomycin. The subculture was performed through 6 to 10 cycles. VSMCs at the logarithmic growth phase in which the growth rate was highest and the cell density was low were obtained on day 4 and day 5 after the inoculation of the cells in the flask.

VSMCs at the logarithmic growth phase were subjected to the hyperthermic treatment for 2 hours in an incubator set at a temperature of 43° C. in each 48-well culture dish (an area of one well is 1 cm²). Subsequently, treated VSMCs were returned to the incubator of 37° C.

The samples were collected just before the hyperthermic treatment, just after the hyperthermic treatment, and on day 1, day 2, day 3, day 4, day 5 and day 6.

### CONTROL EXAMPLE 1

Cells without the hyperthermic treatment were collected from the same VSMCs as in Example 1 at the same points of time to obtain control samples.

### TEST EXAMPLE 1

With respect to the samples in Example 1 and Control Example 1, the number of cells was counted, and CDA-500 grain analyzer manufactured by Cysmex was used in the counting. In the test, the same independent experiment was repeated three times to obtain the same results, and one experiment was performed for three samples (n=3) in each case. The results were shown in FIG. 1 in terms of mean value±standard deviation value.

As is apparent from the curve of the vascular smooth muscle cell growth in FIG. 1, in the sample of Example 1 unlike those of Control Example 1, the increase in number of cells was not substantially identified from just after the hyperthermic treatment to day 1. Even from day 2 onward, the increase in number of cells was considerably inhibited in Example 1 as compared to Control Example 1. The cell density was low, and the growth rate was decreased.

In the samples of Example 1, cells separated from the bottom of the flask were not observed after the hyperthermic treatment. These results revealed that the cell growth of VSMCs was strongly inhibited by the hyperthermic treatment.

### EXAMPLE 2

VSMCs at the logarithmic growth phase in Example 1 were cultured in a low serum medium comprising 0.1% fetal bovine serum (FBS) for 3 days to obtain quiescent smooth muscle cells (quiescent VSMCs) with the growth stopped except cells falling into apoptosis by serum depletion. Quiescent VSMCs were put in a 5% FBS-containing liquid medium, and subjected to a series of steps of performing the growth stimulation to divide cells. The effect of the hyperthermic treatment just after the growth stimulation was first observed. The hyperthermic treatment was conducted at 43° C. for 2 hours. Subsequently, the growth stimulation was performed, and the samples were collected on day 2 and day 5. For examining the preferable timing of the hyperthermic treatment after the growth stimulation, the growth stimulation was performed with 5% FBS at each set time (point of time indicated at mark—in FIG. 2) from 0 to 15 hours, starting from the outset of the hyperthermic treatment to prepare plural samples.

### CONTROL EXAMPLE 2

Control samples were collected at each set time without conducting the hyperthermic treatment in the same manner as in Example 2.

### TEST EXAMPLE 2

With respect to the samples in Example 2 and Control Example 2, the number of cells was counted just after the hyperthermic treatment and on day 2 and day 5 after the growth stimulation. The counting results are shown in FIG. 2. The samples were observed through a phase-contrast microscope just after the hyperthermic treatment and on day 2 and day 5. The phase-contrast micrographs are shown in FIGS. 3(a), 3(b), 3(c), 3(d) and 3(e). (a) is a quiescent VSMCs, (b)(c) are the cells 2 and 5 days passed from stimulation was performed with 5% FBS-containing medium and cultivated without the hyperthermic treatment, (d)(e) are the cells hyperthermic treatment(43, 2 hours) was performed 2 hours after the growth stimulation, and 2 and 5 days passed from growth stimulation.

In Example 2, the effect of the hyperthermic treatment after the growth stimulation was observed using the quiescent smooth muscle cells (quiescent VSMCs) in place of the normal contraction type smooth muscle cells constituting the vascular tunica media for confirming the effect of the hyperthermic treatment in the cell growth experimental system based the mechanism of the restenosis after the coronary intervention.

As is apparent from FIG. 2, regarding the timing of the hyperthermic treatment after the growth stimulation, a significant cell growth inhibition effect was shown in Example 2 as compared to Control Example 2 at any point of time. However, in the observation on day 2 after the growth stimulation, the number of cells tended to be slightly increased in comparison to the number of cells subjected to the hyperthermic treatment at 2 hours after the growth stimulation. From this result, it was found that concerning the hyperthermic treatment after the growth stimulation, the maximum effect was shown in the hyperthermic treatment performed at 2 hours after the growth stimulation in the model of the cell culture experimental system.

According to the observation through the phase-contrast microscope as shown in FIG. 3, the cell density in Control Example 2 becomes high which indicates the decrease in growth potency by hindrance of cell contact ((b)(c)). In contrast, the cells subjected to the hyperthermic treatment in Example 2 still remain low in cell density at this point of time, clearly showing the growth delay((d)(e)).

### EXAMPLE 3

Quiescent VSMCS were previously subjected to the hyperthermic treatment at 43° C. for 2 hours. Subsequently, the growth stimulation was applied thereto with a 5% FBS-containing medium. The timing of the growth stimulation after the hyperthermic treatment was 0, 2, 4, 8 and 12 hours, and plural samples were collected at each point of time.

### CONTROL EXAMPLE 3

The growth stimulation was applied to quiescent VSMCS without the hyperthermic treatment in the same manner as in Example 3 to obtain control samples.

### TEST EXAMPLE 3

With respect to the samples in Example 3 and Control Example 3, the number of cells was counted just after the hyperthermic treatment and on day 2 and day 5 after the growth stimulation. The results are shown in FIG. 4.

In Example 3, the order of the hyperthermic treatment and the growth stimulation in Example 2 was changed. The hyperthermic treatment was first performed (thermal preconditioning), and the growth stimulation was then performed. Thereafter, the growth inhibition effect was observed.

As is clear from FIG. 4, the highest growth inhibition effect was observed in the group to which the growth stimulation was applied simultaneously with the hyperthermic treatment. The earlier the timing of the growth stimulation, the higher level of the growth stimulation was shown. In view of this time relation, the growth inhibition effect can be presumed to differ from a stress resistance acquiring phenomenon of cells by the hyperthermic treatment. The reason is as follows. Ordinarily, in the time zone in which a heat shock protein 70 family is most induced in cells subjected to the hyperthermic treatment, the cells subjected to the hyperthermic treatment secondarily start to show a resistance to stress by ischemia, radical oxygen or the like. It occurs at the earliest after several hours to several tens of hours from the hyperthermic treatment.

### EXAMPLE 4

Quiescent VSMCs were subjected to the growth stimulation, and the samples after 1, 2, 4 and 6 hours were subjected to the hyperthermic treatment. The treatment time was 2 hours, and the temperature was in the range of from 41 to 44° C. The treatment was performed at a rate of rise of 1° C. In the experiment, an incubator set at each temperature was used, and each sample was put in the incubator with a culture flask.

### CONTROL EXAMPLE 4

Control samples without the hyperthermic treatment were obtained in the same manner as in Example 4.

### TEST EXAMPLE 4

With respect to the samples in Example 4 and Control Example 4, the number of cells was counted at each set time. The results are shown in FIGS. 5(a) and 5(b).

In Example 4, the hyperthermic treatment time of the quiescent VSMCs subjected to the growth stimulation was set at 2 hours, and the relation between the preferable treatment time and the treatment timing was examined.

Here, the loss of the cells by separation just after the hyperthermic treatment was not observed at any set temperature shown in FIG. 5. As shown in FIG. 5(a), the growth inhibition effect was identified at 42° C. or more. As shown in FIG. 5(b), at the hyperthermic treatment temperature of 42° C., the decrease in number of cells was identified in the hyperthermic treatment cell group after the lapse of 4 hours from the growth stimulation, but the rate thereof was only approximately 35%. At 43° C., the marked growth inhibition effect was identified regardless of the timing of the hyperthermic treatment. Especially, the hyperthermic treatment at 1 hour and 2 hours from the growth stimulation showed the growth inhibition effect close to the growth stop, which proved a propriety of the results in Example 2. At 44° C., the number of cells was lower than the number of quiescent VSMCs before the growth stimulation at any point of time. This result suggests that the loss by cell death takes part in the cell growth inhibition at 44° C.

The foregoing results show that the hyperthermic treatment temperature at 2 hours after the growth stimulation is preferably set at at least 42° C., and 43° C. at which the effect close to the growth stop is more preferable.

Considering the results of the cell growth inhibition tests in Examples 2, 3 and 4, the timing of the hyperthermic treatment which gives the highest effect of the hyperthermic treatment in the model of the cell culture experimental system is 2 hours from the growth stimulation. The optimum hyperthermic treatment temperature is 43° C.

### EXAMPLE 5

The treatment time was specified at 43° C. Quiescent VSMCs were stimulated with 5% FBS, and samples were collected at 1 hour and 2 hours after the growth stimulation when the growth inhibition effect was high in Example 2. The respective samples were subjected to the hyperthermic treatment for various treatment times, 30 minutes, 60 minutes, 90 minutes, 120 minutes and 180 minutes.

### TEST EXAMPLE 5

With respect to the samples in Example 5, the number of cells was counted. The results are shown in FIGS. 6(a) and 6(b).

In Example 5, the effective treatment time was examined at the hyperthermic treatment temperature of 43° C. As is clear from FIGS. 6(a) and 6(b), the growth inhibition effect at the hyperthermic treatment temperature of 43° C. significantly appeared in the hyperthermic treatment time of 90 minutes or more. The result close to the growth stop was obtained in the hyperthermic treatment time of 120 minutes or more.

### EXAMPLE 6

Quiescent VSMCs in Example 2 were subjected to the hyperthermic treatment at 43° C. for 2 hours, and then returned to an incubator of 37° C. on day 1 and day 2. The incubation was continued in a liquid medium containing 0.1% FBS. The medium was replaced with a 5% FBS-containing liquid medium on day 3 and day 4 to apply the growth stimulation.

### CONTROL EXAMPLE 6

Quiescent VSMCs without the hyperthermic treatment was incubated as in Example 6, and subjected to the growth stimulation to obtain control samples.

### TEST EXAMPLE 6

The cells of the samples were recovered just before the hyperthermic treatment, just after the hyperthermic treatment and on day 1, day 2, day 3 and day 4, and the number of cells was counted. The results are shown in FIG. 7.

In Example 6, it was examined whether the growth inhibition by the hyperthermic treatment of the vascular smooth muscle cells abnormally grown might have an adverse effect on normal contraction type smooth muscle cells constituting the vascular tunica media. Since it is difficult to redifferentiate the culture smooth muscle cells into contraction type smooth muscle cells in the cell culture experimental system, quiescent smooth muscle cells (quiescent VSMCs) similar thereto in growth state were used instead.

As is clear from FIG. 7, in the samples of Example 6, and approximately the same number of cells at any point of time after the hyperthermic treatment as in the control sample of Control Example 6 was shown. Further, separation of the cells from the bottom of the culture flask was not observed. From these results, it was confirmed that quiescent VSMCs had a potency to withstand the hyperthermic treatment and were not brought to cell death.

### EXAMPLE 7

Bovine aortic endothelial cells (BAECs) were collected from the tunica media of the bovine aorta, and subjected to primary culture. BAECs subjected to the primary culture were subcultured in an incubator using a culture flask. The incubator was set at a temperature of 37° C., and filled with humid air containing 5% carbon dioxide. In the subculture, a cell liquid culture medium was replaced every three days. When the culture cell density became high, the cells were recovered with EDTA-trypsin, and inoculated in another flask. The cell growth liquid medium contained 10% FBS, 10 ng/ml of EGF, 100 units/ml of penicillin and 100 mg/ml of streptomycin. The subculture was performed through 12 to 14 cycles. BAECs at the logarithmic growth phase in which the growth rate was highest and the cell density was low were obtained on day 4 and day 5 after the inoculation of the cells in the flask.

BAECs at the logarithmic growth phase were subjected to the hyperthermic treatment for 2 hours in an incubator set at a temperature of 43° C. in each 48-well culture dish (an area of one well is 1 cm²). Subsequently, treated BAECs were returned to the incubator of 37° C. to obtain a sample.

Meanwhile, quiescent BAECS obtained by serum depletion for 3 days were subjected to the foregoing hyperthermic treatment at 43° C. for 2 hours after the lapse of 1 hour or 2 hours from the stimulation with a growth medium to prepare a sample.

### CONTROL EXAMPLE 7

Cells were cultured in an incubator of 37° C. in the same manner as in Example 7 after the cell inoculation without the hyperthermic treatment to obtain a control sample of BAECs.

With respect to quiescent BAECs, a control sample without the hyperthermic treatment was prepared in the same manner as in Example 7.

### TEST EXAMPLE 7

With respect to the samples of BAECs at the growth phase in Example 7 and Control Example 7, the number of cells was counted just before the growth treatment, just after the growth treatment, and on day 1, day 2, day 3 and day 4. The results are shown in FIG. 8(a).

With respect to quiescent BAECs in Example 7 and Control Example 7, the number of cells of the samples just before the hyperthermic treatment and the samples at 1 hour and 2 hours and on day 1 and day 2 after the growth stimulation was counted. The results are shown in FIG. 8(b).

In Example 7, the influence of the hyperthermic treatment on the growth of the vascular endothelial cells was observed as in Example 6.

As is apparent from the cell growth curve in Example 8(a), in BAECs, approximately the same increase in cell number as in the control samples of Control Example 7 was identified in the samples of Example 7. The decrease in cell growth rate observed in the synthesis type smooth muscle cells at the logarithmic growth phase as shown in FIG. 1 was not identified.

As is apparent from FIG. 8(b), the effect of inhibiting the cell growth by the hyperthermic treatment was not identified either on quiescent BAECS.

### EXAMPLE 8

Quiescent VSMCs were subjected to the growth stimulation with 5% FBS. After the lapse of 2 hours, the cells were subjected to the hyperthermic treatment at 43° C. for 2 hours to obtain sample (A). Likewise, the cells were subjected to the hyperthermic treatment at 44° C. for 2 hours to obtain sample (B).

Quiescent VSMCs were subjected to the same hyperthermic treatments without the growth stimulation to obtain samples (C) and (D).

### CONTROL EXAMPLE 8

Control samples without the hyperthermic treatment were prepared as in Example 8.

### TEST EXAMPLE 8

With respect to the samples of Example 8 and Control Example 8, the number of cells was adjusted to approximately 1×10⁶ cells after the recovery. The cells were immobilized on ice for 30 minutes using 70% ethanol on ice to obtain samples for analyzing a cell cycle to measure an intracellular DNA content. The cells were once washed with a liquid medium, and then treated with RNase (100 mg/ml) at 37° C. for 30 minutes to decompose an RNA. Subsequently, 10 mg/ml of propionium iodide was added, and the mixture was treated at room temperature for 15 minutes to stain an intracellular DNA. The cells were centrifuged to remove the supernatant. The resulting cells then refloated and were mixed with 1 ml of a phosphate-buffered saline (PBS), and analyzed with a flow cytometer. The results of sample (A) are shown in FIG. 9(a) and the results of sample (B) in FIG. 9(b).

Likewise, regarding samples (c) and (d), the change with time in the analysis of the cell cycle was examined. The results are shown in FIGS. 10(a) and 10(b).

In Example 8, it was confirmed whether the growth delay of the smooth muscle cells by the hyperthermic treatment (Examples 2 and 3) was ascribable to the stop of the cell cycle progression or to the occurrence of the cell death.

As shown in the histogram of FIG. 9(a), in case of the hyperthermic treatment at 43° C. for 2 hours, the number of cells at S phase and G2/M phase on day 1 after the growth stimulation was clearly decreased in the samples subjected to the hyperthermic treatment in Example 8 as compared to the control examples to show a pattern of a G1 phase stop phenomenon. Further, on day 2 after the growth stimulation, a state of delayed entry into S phase and a sub-G1 population meaning DNA destruction, namely cell death were identified. On day 5 after the growth stimulation, this sub-G1 population disappeared, and the state was not so different from that of the control samples.

As shown in FIG. 9(b), even at the hyperthermic treatment temperature of 44° C., the sub-G1 population started to appear on day 2 after the growth stimulation, and its rate was higher than that at the treatment temperature of 43° C. On day 5 after the growth stimulation, the sub-G1 population was more increased.

Meanwhile, the cells without the growth stimulation, as is clear from FIG. 10(a), thereafter underwent the same progression as in the control samples without the hyperthermic treatment, even though the hyperthermic treatment was performed at 43° C. for 2 hours. Especially on day 2 after the hyperthermic treatment, the sub-G1 population observed in FIG. 9(a) did not appear at all. This histogram indicates that quiescent VSMCs are not brought to death upon withstanding the hyperthermic treatment at 43° C. for 2 hours as in Example 6.

However, as shown in FIG. 10(b), at the treatment temperature of 44° C., the sub-G1 population appeared also on quiescent VSMCs at a high rate on day 2 after the hyperthermic treatment. The appearance of the dead cells shown by this sub-G1 population indicates that the hyperthermic treatment at 44° C. for 2 hours already loses the selectivity based on the cell growth state and is a stress by which to bring even the smooth muscle cells in the cell growth state to cell death.

In a result, the most appropriate temperature to inhibit the overgrowth of the synthesis type smooth muscle cells is 43° C.

### EXAMPLE 9

The following samples were prepared.

Sample 1: VSMCs at the logarithmic growth phase.

Sample 2: Quiescent VSMCs

Sample 3: Control cell group obtained by subjecting quiescent VSMCs to the growth stimulation and culturing the cells for 2 days.

Sample 4: Cells obtained by subjecting quiescent VSMCs to the hyperthermic treatment at 44° C. for 2 hours after 2 hours from the growth stimulation and further culturing the resulting cells for 2 days.

Sample 5: Cells obtained by subjecting VSMCs at the logarithmic growth phase to the UV irradiation for 5 minutes and culturing the resulting cells for 2 days.

Sample 6: Cells obtained by subjecting VSMCs at the logarithmic growth phase to the UV irradiation for 45 minutes.

Sample 7: Cells obtained by performing the extreme osmotic treatment upon replacing a medium with distilled water.

Sample 8: Cells obtained by subjecting quiescent VSMCs to the high temperature treatment at 55° C. for 2 hours after 2 hours from the growth stimulation and culturing the resulting cells for several hours to several tens of hours.

### TEST EXAMPLE 9

Samples 1 to 8 of Example 9 were subjected to the Giemsa staining. The cell form of sample 4 was observed under an optical microscope while comparing it with the forms of the other samples. The results are shown in FIG. 11 to FIG. 14.

In the observation under the optical microscope in Example 9, it was confirmed whether the cells containing approximately 15% of the sub-G1 DNA on day 2 after the growth stimulation by the hyperthermic treatment in Example 8 were based on necrosis or apoptosis as the death mode.

As a result of the microscopic observation, the cells partially observed by being incorporated in the great majority of quiescent VSMCs of sample 2 and the cells remarkably appearing by the UV irradiation of sample 5 for 5 minutes showed reduction in size of a whole cell, agglomeration of a nucleus and fragmentation which are generally recognized as typical phenomena of apoptosis. In contrast, swelling of a whole cell and a nucleus was observed in necrosis cells of samples 6 to 8. The change in form of the cells subjected to the hyperthermic treatment was clearly different from the change in form of the necrosis cells. From the conditions of occurrence of large and small projections, narrowing of a plasma and agglomeration of a nucleus, the mode of death can be presumed to be close to an apoptosis image.

### EXAMPLE 10

The following samples were prepared.

Sample 1: Sample obtained by the DNA extraction from VSMCs at the logarithmic growth phase.

Sample 2: Sample obtained by the DNA extraction from quiescent VSMCs.

Sample 3: Sample obtained by subjecting quiescent VSMCs to the growth stimulation with 5% FBS and, after 1 day, performing the DNA extraction. (Control without the hyperthermic treatment)

Sample 4: Sample obtained by performing the DNA extraction from the same cells as in sample 3 after 2 days. (-ditto-)

Sample 5: Sample obtained by performing the DNA extraction from the same cells as in sample 3 after 5 days. (-ditto-)

Sample 6: Sample obtained by applying the growth stimulation with 5% FBS to quiescent VSMCs, subjecting the cells to the hyperthermic treatment at 44° C. after 2 hours of the growth stimulation, and performing the DNA extraction from the cell group on day 1 after the growth stimulation.

Sample 7: Sample obtained by the DNA extraction from the cell group on day 2 after the growth stimulation of the same cells subjected to the hyperthermic treatment as in sample 6.

Sample 8: Sample obtained by the DNA extraction from the cell group on day 5 after the growth stimulation of the same cells subjected to the hyperthermic treatment as in sample 6.

Sample 9: Sample obtained by subjecting VSMCs at the logarithmic growth phase to the UV irradiation for 5 minutes and performing the DNA extraction from the cell group on day 1 after the irradiation.

Sample 10: Sample obtained by conducting the same UV treatment as in sample 9 and performing the DNA extraction from the cell group on day 2 after the irradiation.

Sample 11: Sample obtained by conducting the same UV treatment as in sample 9 and performing the DNA extraction from the cell group on day 3 after the irradiation.

Sample 12: Sample obtained by conducting the same UV treatment as in sample 9 and performing the DNA extraction from the cell group on day 5 after the irradiation.

### TEST EXAMPLE 10

The DNA extraction from the respective samples and the agarose electrophoresis were performed according to an apoptosis ladder detection method (Apoptosis Ladder Detection Kit Wako). Specifically, concerning the respective samples, the DNA extraction was performed from 1×10⁶ cells through isopropanol precipitation. The extracted DNA was dissolved in 50 ml of a buffer (TE buffer) solution to prepare a sample. Then, 20 ml of the sample was subjected to the agarose electrophoresis.

In the detection of the DNA in the gel after the electrophoresis, staining was performed in the dark for 30 minutes using ethidium bromide at a final concentration of 0.5 mg/ml. Subsequently, photographs were taken using a UV transluminator. The results are shown in FIG. 15.

In Example 10, it was difficult to judge whether the mode of death is apoptosis or not by the mere morphological estimation of Example 9. Accordingly, the phase-contrast microscopic observation by DNA ladder detection was used.

In lanes 1 to 8 of FIG. 15, the DNA excision pattern was analyzed by the agarose gel electrophoresis. The DNA extract obtained from the cells subjected to the hyperthermic treatment shows fragmentation (DNA ladder) in a nucleosome unit. This phenomenon was clearly observed in sample 8 obtained by the DNA extraction from the cell group on day 5 after the growth stimulation. As a result of the DNA ladder detection, it was found that the mode of death induced by the hyperthermic treatment at 44° C. was based on the apoptosis mechanism.

Lanes 9 to 12 show excision patterns of DNAs subjected to the agarose gel electrophoresis on samples 9 to 12 (on day 1, day 2, day 3 and day 5 after the UV irradiation). These are typical patterns of the apoptosis in this experimental method.

### EXAMPLE 11

The following samples were prepared.

Sample 1: VSMCs at the logarithmic growth phase.

Sample 2: Quiescent VSMCs.

Sample 3: Control cells obtained by subjecting quiescent VSMCs to the growth stimulation with 5% FBS and then culturing the cells for 2 days.

Sample 4: VSMCs obtained by subjecting quiescent VSMCs to the hyperthermic treatment at 43° C. for 2 hours after 2 hours from the growth stimulation and further culturing the resulting cells for 2 days.

Sample 5: Cell group obtained by subjecting VSMCs at the logarithmic growth phase to the UV irradiation for 5 minutes and then culturing the resulting cells for 2 days.

### TEST EXAMPLE 11

With respect to the samples in Example 11, the staining experiment was performed by the Terminal Deoxynucleotidyltransferase (TdT)-mediated DdTP-biotin Nick End Labeling method (TUNEL).

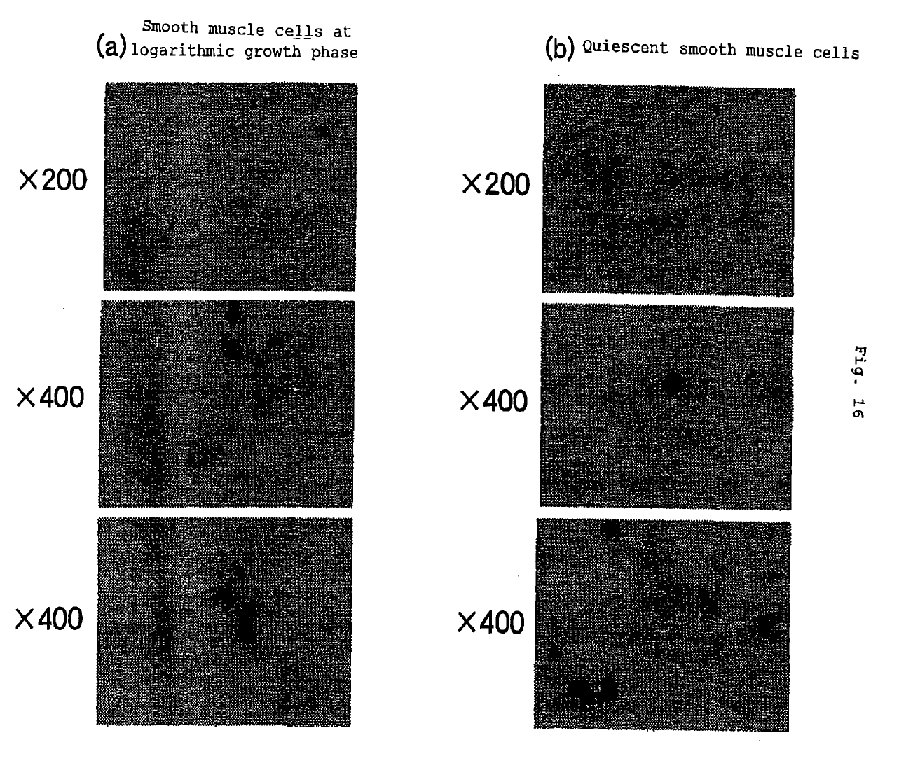

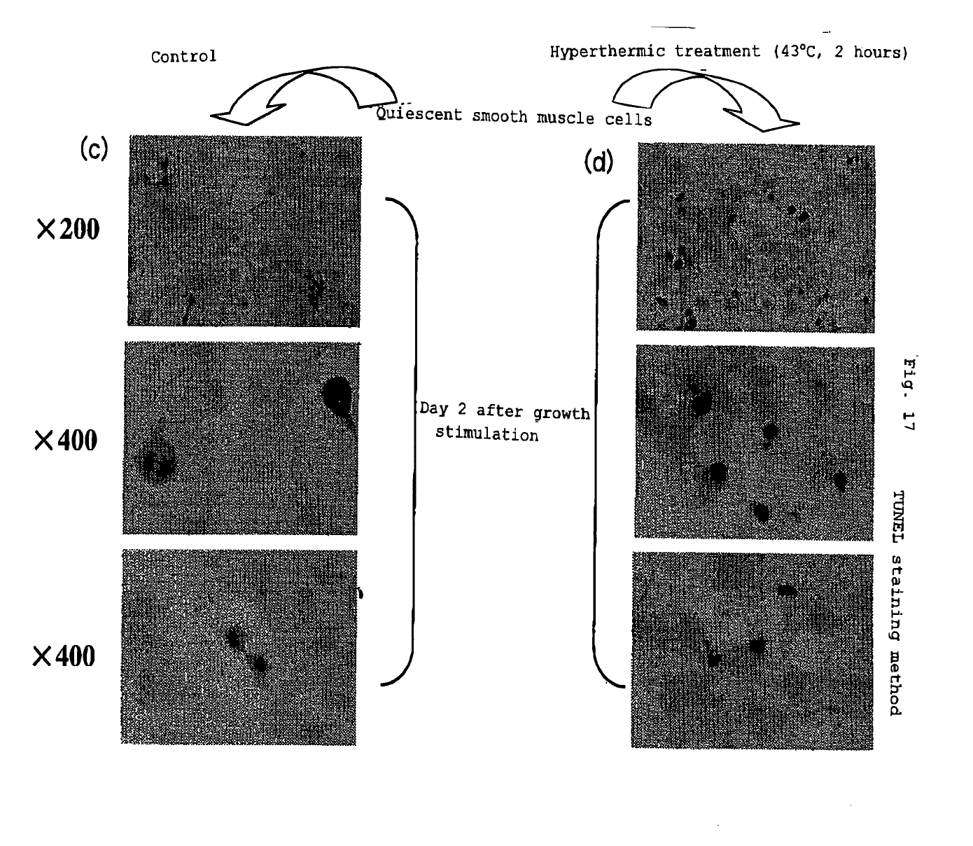

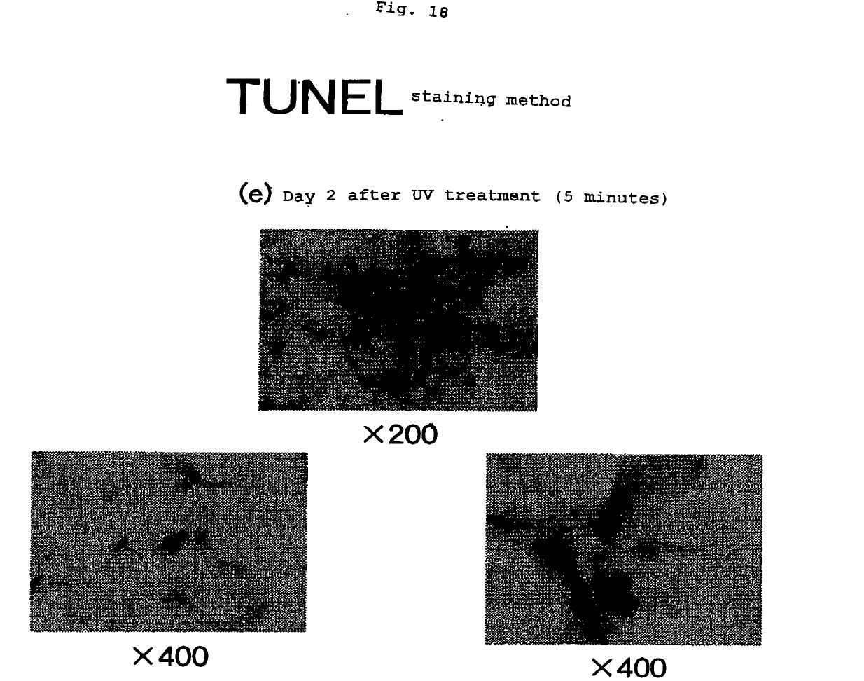

First, the cells inoculated on glass chamber slides were subjected to various experiments, and then immobilized with 4% formaldehyde for 10 minutes. Subsequently, the staining was performed according to a protocol of Apoptosis in situ Detection Kit Wako. The stained cells were observed through an optical microscope. The results are shown in FIG. 16 to FIG. 18.

From the results in Example 9 and Example 10, the mode of cell death induced by the hyperthermic treatment at 43° C. for 2 hours can be presumed to be close to apoptosis. For further proving this, the staining experiment was performed by the TUNEL method in Example 11.

As a result, in the cell group obtained by subjecting quiescent VSMCs of sample 4 to the growth stimulation with 5% FBS and applying the hyperthermic treatment at 43° C. for 2 hours after 2 hours from the growth stimulation, the cell death of approximately 15% indicated TUNEL-positive as shown in FIG. 17(d). Consequently, this cell death is presumably based on the apoptosis mechanism.

### EXAMPLE 12

The following samples were prepared.

Sample 1: VSMCs at the logarithmic growth phase.

Sample 2: Quiescent VSMCs.

Sample 3: Cell group obtained by stimulating quiescent VSMCs with 5% FBS and subjecting the cells to the hyperthermic treatment at 44° C. for 2 hours to induce apoptosis cells.

Sample 4: Apoptosis induction cell group which is the same as in sample 3 except that the hyperthermic treatment temperature is 43° C.

Sample 5: VSMCs apoptosis cell group induced by the UV irradiation for 5 minutes.

Sample 6: Cells obtained by subjecting quiescent VSMCs to the growth stimulation with 5% FBS and conducting the high-temperature treatment at 55° C.

### TEST EXAMPLE 12

Sample 3 of VSMCs subjected to the high-temperature treatment in Example 12 was analyzed with a flow cytometer using anexxin V-FITC/PI.

The exposure of an anexxin V binding site (phosphatidylserine) on the cell membrane surface was detected according to a protocol of Annexin V-FITC Kit (manufactured by Immunotech A Beckman Coulter Company).

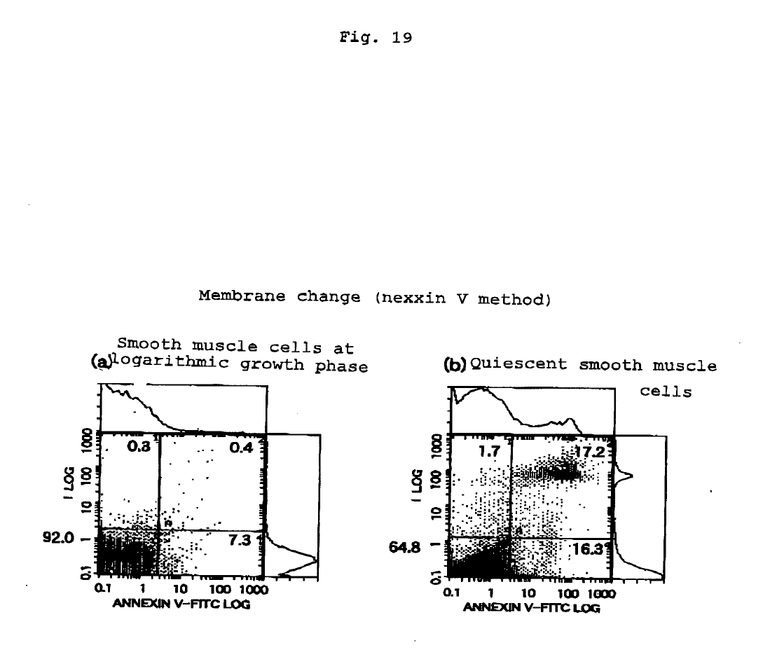

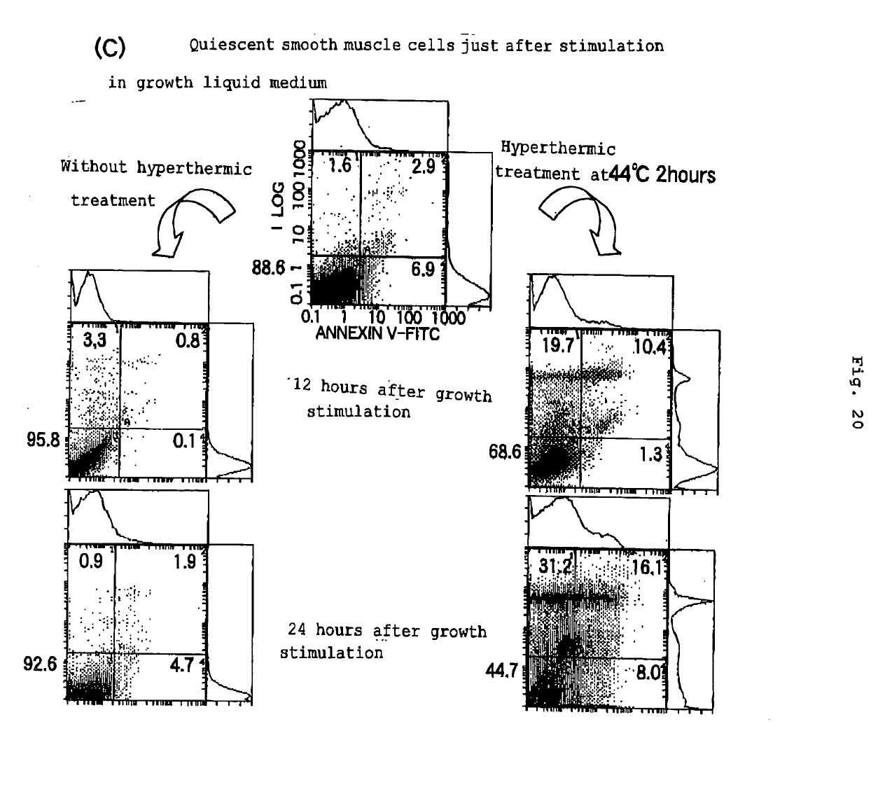

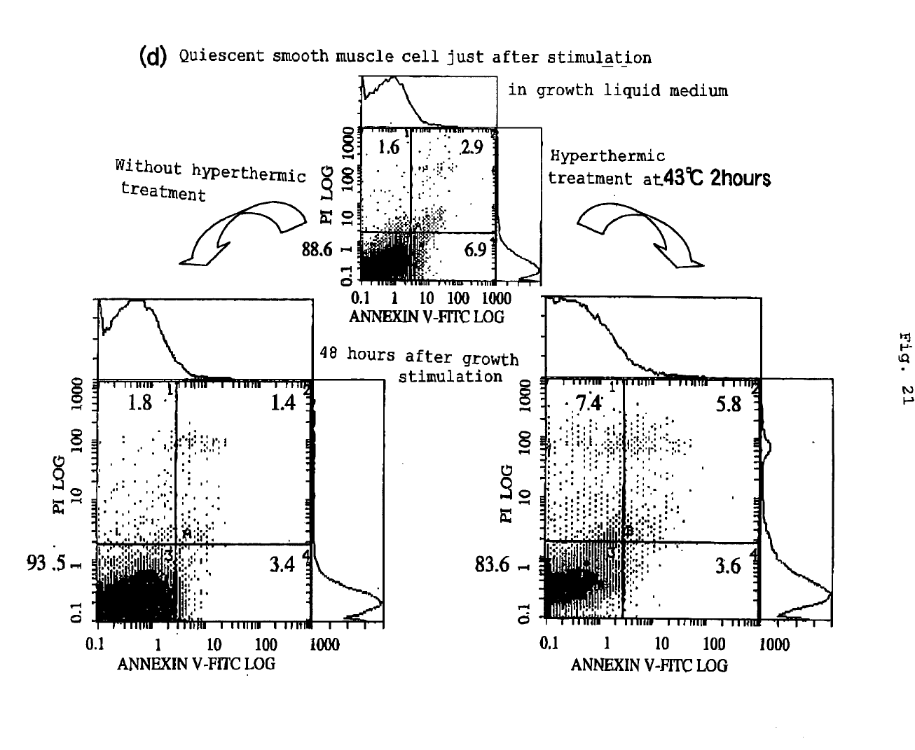

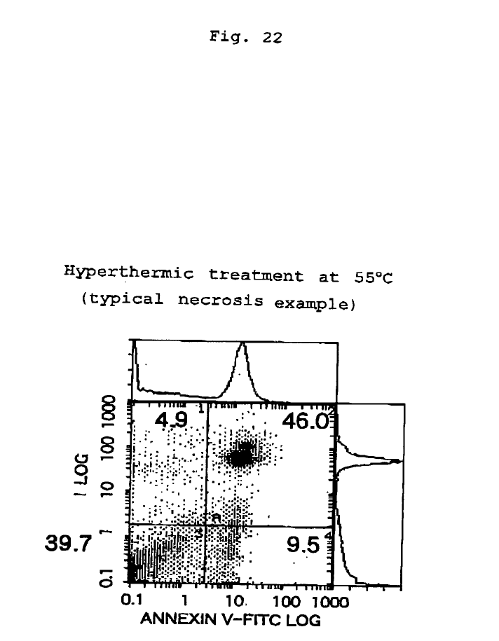

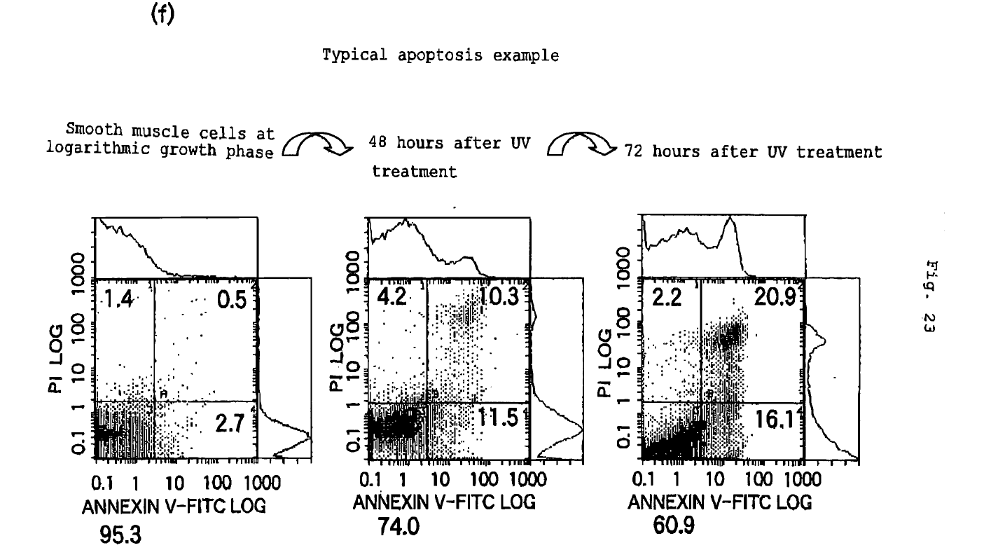

That is, 1×10 pieces of each cell sample was separated with a centrifugal separator for 5 minutes, and then washed once using a liquid medium of 4° C. Subsequently, the cells were caused to float in a combined buffer attached, and 5 ml of an annexin V-FITCC solution and 5 ml of propidium iodide PI (250 mg/ml) were added thereto. They were gently mixed with stirring for a reaction. The reaction mixture was allowed to stand still in the dark on ice for 10 minutes, and then analyzed using a flow cytometer. The results are shown in FIGS. 19 to FIG. 23.

From the results in Examples 9 to 11, the change in mode of the cell death induced by the hyperthermic treatment is considered to be based on the apoptosis mechanism. Therefore, in Example 12, it was examined whether the exposure of phosphatidylserine on the cell membrane surface as an annexin V binding site which is generally identified in the initial stage of the apoptosis cell death occurred or not.

As is apparent from FIGS. 19(b), the apoptosis cells in the early stage shifting to a distribution region (region 4) that shows PI-negative and annexin V-FITC-positive meaning viable cells, or necrosis cells shifting to a distribution region (region 2) that shows PI-positive and annexin V-FITC-positive were confirmed in both of samples 1 and 2. Further, as shown in FIGS. 20(c) and 20(d), in both of sample 3 (hyperthermic treatment at 44° C.) and sample 4 (hyperthermic treatment at 43° C.), more than half of the cells that showed propidium iodide-positive meaning dead cells, showed annexin V-negative, were shifted from the viable cell distribution region 3 to the region 1, and showed PI-positive. This was quite a peculiar characteristic of the vascular smooth muscle apoptosis cells induced by the hyperthermic treatment. It was not reported in the ordinary apoptosis cells, nor was it identified at all in the typical necrosis cells. The rates at which the viable cells were shifted from the region 3 to the region 1 at the hyperthermic treatment temperatures of 44° C. and 43° C. were 31.2% and 7.4% at 24 hours after the growth stimulation, and the rates of the dead cells in combination with those in the region 2 were 31.2%+16.1% and 7.4%+5.8% respectively. Further, the typical secondary necrosis of the apoptosis cells was observed in samples 5 and 6 in FIGS. 22(e) and 22(f).

The final conclusion in the culture cell experimental systems in Examples 1 to 12 was summarized as follows.

(1) When the synthesis type smooth muscle cells at the logarithmic growth phase were subjected to, for example, the hyperthermic treatment at 43° C. for 2 hours after 2 hours from the growth stimulation, the growth of the smooth muscle cells was inhibited most efficiently.

(2) The same hyperthermic treatment does not bring the quiescent smooth muscle cells of the experimental culture system equivalent to the normal contraction type smooth muscle cells to the cell death.

(3) The same hyperthermic treatment causes almost no growth inhibition on the bovine aortic endothelial cells of the experimental culture cell system equivalent to the vascular endothelial cells either during the growth response or during the growth.

In view of the foregoing and from the standpoint of the cell selective and cell state selective effects generally required in the clinical therapy of cancers based on the cytotoxicity, the cell growth inhibition phenomenon by the hyperthermic treatment has the cell selectivity for the G1 phase stop phenomenon and the cell selectivity and the cell state selectivity for the apoptosis induction.

These Examples prove the usefulness and the safety of the hyperthermia at a cell level by strictly determining the heating conditions of the hyperthermic treatment upon assuming the clinical application as a method for inhibiting the restenosis after the coronary angioplasty.

INDUSTRIAL APPLICABILITY

According to the invention, the growth of the synthesis type smooth muscle in the vascular subendothelial cavity can be inhibited surely by subjecting the vascular smooth muscle cells to the predetermined hyperthermic treatment before or after the growth stimulation thereof without damaging the surrounding important cells such as normal contraction type smooth muscle cells and vascular endothelial cells constituting the tunica intima of the vascular wall. This growth inhibition effect can be widely applied clinically to vascular lesions for prevention of restenosis after coronary angioplasty and the like as the safe and effective hyperthermia from the aspects of the cell-selective G1 phase stop and the cell-selective and cell condition-selective apoptosis induction.

1. A method for inhibiting growth of vascular smooth muscle cells, characterized by comprising subjecting vascular smooth muscle cells which has responded to growth to the hyperthermic treatment.

2. The method for inhibiting growth of vascular smooth muscle cells as claimed in claim 1, characterized in that the timing of the hyperthermic treatment is any timing selected from 12 hours before growth stimulation of the vascular smooth muscle to 15 hours after the stimulation including the point of time of the stimulation.

3. The method for inhibiting growth of vascular smooth muscle cells as claimed in claim 1, characterized in that the timing of the hyperthermic treatment is after the growth stimulation of the vascular smooth muscle.

4. The method for inhibiting growth of vascular smooth muscle cells as claimed in claim 1, characterized in that the timing of the hyperthermic treatment is 2 hours after the growth stimulation of the vascular smooth muscle.

5. The method for inhibiting growth of vascular smooth muscle cells as claimed in claim 1, characterized in that the hyperthermic treatment temperature is in the range of from 42 to 44° C.

6. The method for inhibiting growth of vascular smooth muscle cells as claimed in claim 1, characterized in that the hyperthermic treatment temperature is in the range of from 43 to 44° C.

7. The method for inhibiting growth of vascular smooth muscle cells as claimed in claim 1, characterized in that the hyperthermic treatment time is in the range of from 90 to 180 minutes.

8. A method for inhibiting growth of vascular smooth muscle cells, characterized by comprising applying the hyperthermic treatment at 43° C. for 2 hours to the vascular smooth muscle cells after 2 hours from the growth stimulation.
